(19) United States
(12) Plant Patent Application Publication
(10) Pub. No.: US 2004/0181840 P1
(43) Pub. Date: Sep. 16, 2004

(54) Dieffenbachia hybrid called "GoldRush"

(76) Inventor: Richard J. Henny, Apopka, FL (US)

Correspondence Address: Thomas D. Stadsklev, Florida Foundation Seed Producers, Inc., P.O. box 309, Greenwood, FL, 32443, US

(21) Appl. No.: 10/386,176

(22) Filed: Mar. 11, 2003

(51) Int. Cl. A01H 5/00

(52) U.S. Cl. PLT/378

(57) ABSTRACT

A new and distinct cultivar of Dieffenbachia that is characterized by large undulating leaves that are deep golden yellow and are highlighted by a dark green border and white midrib with petioles that hold the leaf blade close to the main stem to give a compact appearance on plants that average 3-4 basal shoots.

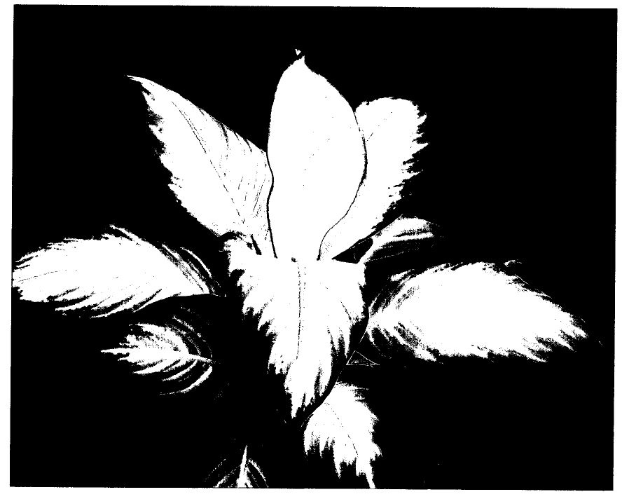

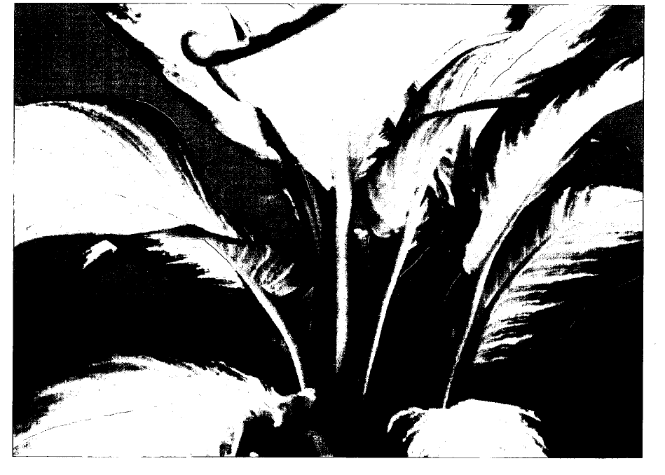

The present invention comprises a new and distinct cultivar of Dieffenbachia, botanically known as Dieffenbachia hybrida, and referred to by the cultivar name ‘GoldRush’.

The new cultivar is a product of a planned breeding program carried out by the inventor Richard Henny in Apopka, Fla. Dr. Henny has performed many crosses with Dieffenbachia since 1980 as part of his extensive ornamental tropical foliage plant breeding program.

The new cultivar is a product of cross-pollination between Dieffenbachia ‘Victory’ (unpatented, pollen parent) and Dieffenbachia ‘Tropic Marianne’ (U.S. Plant Pat. No. 8,832; seed parent).

The cultivar was discovered as a single plant from the progeny of the stated cross by Richard J. Henny in Apopka, Fla. It was selected because of its vigorous growth, fairly large size, compact habit and undulating leaves that displayed a bright golden color accented by a dark green border. Asexual propagation by cuttings, first performed by Richard J. Henny in Apopka, Fla., was used to increase the number of plants for evaluation. Subsequently, plants were propagated by tissue culture of shoot tips that also has demonstrated the stability of the combination of characteristics of ‘GoldRush’ from generation to generation.

The following observations, measurements and values describe plants grown in Apopka, Fla., under greenhouse conditions which closely approximate those generally used in horticultural practice. The following traits have been repeatedly observed to be characteristics that in combination distinguish GoldRush from other Dieffenbachia of the same general type such as the well-known cultivars ‘Tropic Marianne’ (U.S. Plant Pat. No. 8,832) and ‘Camille’ (unpatented).

‘GoldRush’ differs from ‘Victory’ in the following ways.

1. The leaves of ‘Victory’ contain many isolated green flecks or islands of different sizes and shapes against a light greenish-gold background throughout the entire leaf surface. The islands, ranging in size from 0.1 to 1.5 cm in diameter, blend into the leaf edge forming a solid green border. ‘GoldRush’ leaves do not contain any such green islands.

‘GoldRush’ differs from ‘Tropic Marianne’ in the following ways.

1. The leaves of ‘GoldRush’ are very thick with sunken veins that produce a wavy but uniform texture. ‘Tropic Marianne’ leaves are smoother.

2. There are one to two distinct large undulations in ‘GoldRush’ leaf margins that result in a wavy effect. ‘Tropic Marianne’ leaves lack such undulation.

3. The petiole wings on the main shoot of ‘GoldRush’ extend all the way to the base of the leaf blade to give a very compact appearance as the leaf blade hugs the main stem. ‘Tropic Marianne’ petiole wings stop 1-2 cm short of the base of the leaf blade.

4. Leaves on the main stem of ‘GoldRush’ are held closer to the stem to give a more compact appearance compared to ‘Tropic Marianne’.

‘GoldRush’ differs from ‘Camille’ in the following ways.

1. ‘GoldRush’ is much larger and more vigorous than ‘Camille’.

2. ‘GoldRush’ leaves are thicker and much bigger than ‘Camille’.

3. ‘GoldRush’ does not branch as much as Camille’.

‘GoldRush’ differs from ‘Tropic Honey’ in the following ways.

1. ‘GoldRush’ leaves average 8-10 cm longer and 2-4 cm wider than ‘Tropic Honey’ on plants grown in 20 cm diameter pots.

2. ‘GoldRush’ leaves have 1-2 large undulations while ‘Tropic Honey’ has several smaller undulations along the leaf margin.

3. The leaf midrib of ‘GoldRush’ is white while ‘Tropic Honey’ is mostly green.

4. ‘GoldRush’ averages 3-4 basal shoots per plant while ‘Tropic Honey’ averages 6 or more.

All color references are measured against The Royal Horticultural Society Color Chart. Colors are approximate as color depends on horticultural practices such as light level and fertilization rate, among others, without, however any variance in genotype.

### BRIEF DESCRIPTION OF THE DRAWINGS

The two color photographic drawings were taken from a typical plant of Dieffenbachia ‘GoldRush’ that was 36 weeks old and grown in a 20 cm diameter pot (3.9 liter volume) under appropriate growing conditions. Colors are as accurate as possible with color illustrations of this type.

1. The first drawing depicts a top perspective view of a plant of ‘GoldRush’ showing the golden foliar variegation and the compact foliage canopy due to the short thick clasping petioles. The large undulation in the leaf margins is visible as well as the arching form of the leaves.

2. The second drawing depicts the upper and lower leaf surface and the main stem and petiole coloration.

Origin: Seedling selected from a cross of Dieffenbachia ‘Victory’ (not patented, pollen parent) and Dieffenbachia ‘Tropic Marianne’ (U.S. Plant Pat. No. 8,832; seed parent).

Classification: Dieffenbachia cultivar ‘GoldRush’.

Propagation: Asexual propagation either by tissue culture, stem cuttings or division. Tissue culture propagation involves culturing apical buds that develop multiple lateral shoots that are then harvested, rooted and grown out in a greenhouse.

Plant: The following is a description of a typical 8-month-old plant of Dieffenbachia ‘GoldRush’ growing in a 20 cm diameter pot for under appropriate growing conditions, ‘GoldRush’ has a canopy height, from the soil surface of 29 to 32 cm and a canopy width of 47 to 51 cm. Plants of this age average 3 to 4 basal shoots. The petioles remain closely aligned to the main stem and result in a very compact appearance for the foliage canopy compared to other dieffenbachia cultivars of similar size.

Stem:

Growth pattern.—The stem is erect in growth and is 2.5 to 3.0 cm in diameter five (5) cm above the soil surface. Internode distance is approximately 1.9 cm to 2.2 cm at five (5) cm above the soil.

Color.—The stem color is green 137B/C. The color is uniform with no mottling. Stem areas exposed by petiole removal are yellow green 146B/C. This coloration will gradually darken once exposed to light.

Petiole: The following information is based on the 3rd expanded leaf from the apex on the central stem.

Dimensions.—The petioles are approximately 10.0 to 14.0 cm in length. The petiole has fleshy edges extending from the midrib and referred to as wings. The wings are approximately 8 mm to 12 mm wide one-half the distance from the petiole base to the wing apex. The wings extend from the base of the petiole all the way to the leaf base. The petiole follows the stem axis to within 1.0 to 2.0 cm of the leaf base. The petiole and wings average approximately 7 mm to 10 mm in diameter one-half the distance between the top of the wing and the base of the leaf.

Color.—If looking from above, the petiole and petiole wings are green 137 B/C on the outer edges extending inward about ⅓ the petiole width from each edge. The central portion of the petiole is lighter yellow-green 139D. The petiole underside is green white 157A in a central band approximately 1 cm wide. This stripe darkens on the outer edges to yellow green 146B.

Leaf:

Growth pattern.—The leaf is oblong with a cuspidate apex and cordate base. The margin is entire. The leaf is asymmetric with the side of the leaf unrolling first having less surface area than the side unrolling last. The leaf is positioned parallel to the stem axis at the time of full unrolling, changing to approximately 40-50 degrees above perpendicular to the stem axis as more leaves unroll above it. The midrib is straight over two-thirds the length of the leaf and curved downward toward the tip. The leaf blade displays 1 to 2 distinct undulations along the margin. A single leaf undulation may extend for 8-12 cm from edge to edge with depth of 1-1.5 cm from the lowest point to the highest (determined by laying a straight measuring stick along the leaf margin). The leaf is thick and somewhat puckered.

Dimensions.—For the pot size and growing time indicated, the largest leaf is approximately 36-38 cm long and approximately 14-17 cm wide. An average sized leaf is approximately 35-36 cm long and approximately 14-15 cm wide.

Midrib.—The leaf midrib is thick and prominent and ranges from a width of 1 cm at the base of the leaf to approximately 0.7 to 0.8 cm at a distance equally between the leaf base and the leaf tip. The leaf midrib is white 155A.

Primary veins.—The primary veins are sunken into the upper leaf surface and protrude from the lower surface. The primary veins are the same yellow-green color (145 B/C) as the center of the leaf.

Color and pattern.—Leaf color pattern is uniform beginning with a green 139A border that is 1.0 to 2.0 cm in width edges mature leaf blades on the upper and lower surfaces. The central leaf area is a uniform yellow-green 145B/C. The leaf midrib is white 155A.

Axillary breaks: In our experimental growth tests plants produced averaged 3-4 axilliary breaks with at least one leaf expanded.

Inflorescence: Plants have not yet been observed in flower. However, they are expected to be typical of Dieffenbachia and do not have commercial significance.

Roots: Moderately thick white roots with fine laterals.

Disease resistance: Dieffenbachia ‘GoldRush’ has no special disease or insect resistance compared to other Dieffenbachia cultivars.

I claim:

1. A new and distinct Dieffenbachia hybrid characterized by large undulating leaves that are deep golden yellow and are highlighted by a dark green border and white midrib with petioles that hold the leaf blade close to the main stem to give a compact appearance on plants that average 3-4 basal shoots.
